(19) United States
(12) Patent Application Publication
(10) Pub. No.: US 2024/0350520 A1
(43) Pub. Date: Oct. 24, 2024

(54) Method to Stimulate the Growth of Veillonella in Human Gut and Increase Athletic Performance

(71) Applicant: Zhong Zhang, Ithaca, NY (US)

(72) Inventor: Zhong Zhang, Ithaca, NY (US)

(73) Assignee: Mellitas Health Foods, LLC, (omitted)

(21) Appl. No.: 18/303,033

(22) Filed: Apr. 19, 2023

(51) Int. Cl. A61K 31/702; A61K 9/00

(52) U.S. Cl. CPC A61K 31/702; A61K 9/0053

(57) ABSTRACT

A method for substantially increasing the level of Veillonella in human gut is described. The method involves taking a type of human milk oligosaccharide, i.e., 2′-Fucosyllactose, orally to increase the level of Veillonella in the human gut. The daily supplementing dosage of 2′-Fucosyllactose is between 2.0 to 4.5 g. The supplementing period is between 20 days and 60 days.

### BACKGROUND OF THE INVENTION

The human gut microbiome is a complex ecosystem that houses trillions of microorganisms, including bacteria, fungi, viruses, and protozoa. These microbes are known to play a crucial role in human health and have been linked to various physiological processes, such as digestion, immunity, and metabolism (1). Recently, research has begun to explore the potential connection between the gut microbiome and athletic performance, with Veillonella emerging as a bacterium of particular interest (2).

Veillonella is a genus of gram-negative anaerobic bacteria commonly found in the oral cavity and gastrointestinal tract (3). This bacterium attracted the attention of researchers when a study published in Nature Medicine in 2019 reported a higher abundance of Veillonella in the gut microbiomes of elite athletes compared to sedentary individuals (4). The study, conducted by Scheiman et al., analyzed the gut microbiomes of 15 Boston Marathon runners and 10 non-runner controls, identifying a significantly higher presence of Veillonella atypica in the athletes.

The researchers hypothesized that Veillonella might enhance athletic performance by metabolizing lactic acid, a byproduct of muscle activity that accumulates during exercise and contributes to muscle fatigue (5). Veillonella can convert lactic acid into propionate, a short-chain fatty acid that serves as an energy source for the body (6). To test this hypothesis, Scheiman et al. isolated Veillonella atypica from one of the athletes and used it to colonize the guts of mice. The mice were then subjected to a treadmill test, and those with elevated Veillonella levels demonstrated a 13% increase in running time compared to the control group (4). The Veillonella and athletic performance are correlated (7).

Other studies have also explored the potential benefits of Veillonella in the context of exercise. A 2020 study published in the Journal of Applied Microbiology found that supplementation with Veillonella parvula, another species of the Veillonella genus, improved exercise capacity and reduced inflammation in mice (8).

The link between Veillonella and athletic performance is an exciting area of research that has the potential to expand our understanding of the gut microbiome's role in human health and performance. While early findings suggest a possible connection between Veillonella and improved exercise capacity, more research are ongoing to establish causality and investigate the underlying mechanisms.

It is possible to supplement live Veillonella and increase the number of Veillonella in human gut, but Veillonella is a strictly anaerobic, which make it hard to produce. A more feasible way to increase the Veillonella in human gut is to feed the gut with food for Veillonella, which help stimulate the growth of Veillonella. It remained a mystery which food

### SUMMARY OF THE INVENTION

This summary is provided solely as an introduction to subject matter that is fully described in the detailed description and drawings. The summary should not be considered to describe essential features nor be used to determine the scope of the claims. Moreover, it is to be understood that both the summary and the detailed description are examples and explanatory only and are not necessarily restrictive of the subject matter claimed.

A method for increasing the Veillonella in human gut using 2′-Fucosyllactose is disclosed which results in a substantial growth of Veillonella in human gut. The method preferably involves taking 2′-Fucosyllactose for a period of 20 days to 60 days orally.

In one embodiment, the 2′-Fucosyllactose is highly purified and taken at a dosage of 2000 mg per day. In other embodiments the 2′-Fucosyllactose is highly purified, with a purity higher than >90%, and taken at a dosage of 2000-4500 mg per day.

In another embodiment, the Veillonella in human gut is increased substantially after taking the 2′-Fucosyllactose.

In another embodiment, a treatment to enhance the athletic performance through taking 2′-fucosyllactose is disclosed herein.

### BRIEF DESCRIPTION OF DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1 is a figure showing the average level of Veillonella level for 4 human subjects before and after taking 2′-Fucosyllactose.

### DETAILED DESCRIPTION OF THE INVENTION

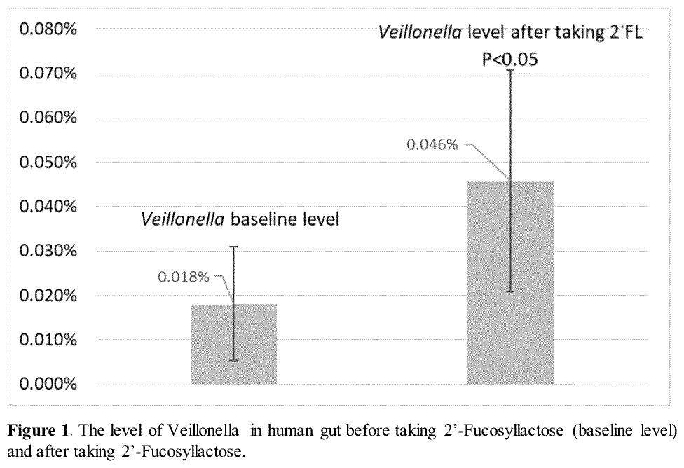

FIG. 1 shows the average level of Veillonella in the studied subjects before and after taking 2′-Fucosyllactose, respectively. The genus, Veillonella, were quantified through the fecal samples collected by the human subjects.

16S ribosomal RNA (rRNA) sequencing was used to quantify the relative abundance of Veillonella in the human fecal samples.

Veillonella levels were expressed as a percentage of total gut bacteria. Veillonella levels were quantified before the supplementation of 2′-Fucosyllactose and also after the supplementation of 2′-Fucosyllactose.

The human subjects were asked to take 2′-Fucosyllactose daily doses ranging from 2000 mg to 4500 mg for 20-60 days with liquid and not to change their regular diet during the period.

The supplementation of 2′-Fucosyllactose into the human subjects' diet for 30-45 days significantly increased the level of Veillonella in their gut, as shown in the FIG. 1. The level Veillonella boosted from 0.018% before the 2′-Fucosyllactose intake to 0.046% after the 2′-Fucosyllactose intake (P<0.05).

### Example

The examples below serve to further illustrate the present disclosure, to provide those of ordinary skill in the art with a complete disclosure and description of how the compounds, compositions, articles, devices, and/or methods claimed herein are made and evaluated, and are not intended to limit the scope of the present disclosure. The examples are not intended to restrict the scope of the present disclosure.

Four healthy subjects were asked to test their fecal samples and determine the baseline level of Veillonella before taking the 2′-Fucosyllactose. After collecting their initial fecal samples, they were instructed to take 2′-Fucosyllactose daily at doses between 2000 mg-4500 mg per day with liquid and not change their regular diet.

The subject was asked to stop the supplementation of 2′-Fucosyllactose after 20-60 days. The subjects were instructed to collect their fecal samples immediately after stopping the supplementation and the fecal samples were tested thereafter.

The levels of Veillonella in the fecal samples were quantified by 16S ribosomal RNA (rRNA) sequencing.

The supplementation of 2′-Fucosyllactose at daily dosages of 2000-4500 mg significantly increased the level of Veillonella in the human gut after 20-60 days.

1. A method for significantly increasing the level of Veillonella in human gut by orally supplementing with 2′-Fucosyllactose.

2. The method of claim 1 wherein the daily supplementation of 2′-Fucosyllactose ranges from 0.1 g to 20 g, preferably between 2 g and 4.5 g.

3. The method of claim 2 wherein the supplementation period ranges from 1 to 100 days, preferably between 20 days to 60 days.
